(19) United States
(12) Patent Application Publication
(10) Pub. No.: US 2008/0003665 A1
(43) Pub. Date: Jan. 3, 2008

(54) Methods and systems for detecting particles

(76) Inventors: Radislav Alexandrovich Potyrailo, Niskayuna, NY (US); Steven Francis LeBoeuf, Schenectady, NY (US); Rui Chen, Niskayuna, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/480,184

(22) Filed: Jul. 3, 2006

(51) Int. Cl. G01N 33/555; C12M 3/00; G01J 3/45

(52) U.S. Cl. 435/287.2; 436/522; 356/451

(57) ABSTRACT

A system for detecting a particle disposed in a detection area. The system includes a light-emitting source for generating light. The light is directed at the particle. The system further includes a modulator configured to in-situ modulate at least one environmental parameter of the particle to alter a detectable response of the particle. The modulator provides an enhancement in detection selectivity of the particle in the presence of interfering particles and species. Further, the system includes a detector configured to detect alteration in the detectable response of the particle.

### STATEMENT REGARDING FEDERALLY SPONSORED RESEARCH & DEVELOPMENT

This invention was made with Government support under contract number W91CRB-04-C-0063 awarded by the United States Army RDECOM Acquisition Center, Aberdeen Proving Grounds, for the Technical Support Working Group. The Government has certain rights in the invention.

### BACKGROUND

The invention relates generally to methods and systems for detecting particles. More particularly, the invention relates to methods and systems for detecting biological particles.

Microorganisms are naturally aerosolized in the atmosphere, and may be a burden to downwind entities. For example, the aerosolized microorganisms may result in respiratory problems. Determining the size of particles may assist in identifying whether the particles are respirable or not. Particle counters also are used in the semiconductor industry to monitor air cleanliness for the particle-sensitive photolithography step. By measuring the absorption of certain optical wavelengths, one also can measure the presence of specific chemicals, such as NOₓ, CO₂, or carbon monoxide.

Conventional approaches involve measuring size characteristics of biological particles in air to differentiate them from ambient material. In this approach, the particle size characteristics between unknown biological aerosols and background material may be compared. For example, the particle size may be estimated by time-of-flight information derived from scattered light. Fourier-transform infrared spectroscopy (FTIR) detection can be used to identify the presence of ice and water vapor.

Further, air-borne particles may be subjected to a light source capable of inducing an emission of fluorescence from the particles. Fluorescence spectroscopy is now widely applied for detection of biological material via analysis of native fluorescence of biomaterials known also as biofluorescence or autofluorescence. For example, fluorescence detected in a range of from about 400 nanometers to about 540 nanometers signals the presence of nicotinamide adenine dinucleotide hydrogen (NADH), which is indicative of biological activity or viability. Such a fluorescence-based technique generates data from certain molecular components of biological material, allowing it to be a tool for nonspecific agent detection.

Unfortunately, the intrinsic fluorescence bands from biological materials are relatively spectrally wide; the primary fluorophores in the majority of bioaerosols fall into only a few broad categories. These include the aromatic amino acids, tryptophan, tyrosine, phenylalanine, nicotinamide adenine dinucleotide compounds, flavins, chlorophylls, and others.

### BRIEF DESCRIPTION OF THE DRAWINGS

FIG. 1 is a diagrammatical illustration of a particle detection system in accordance with an exemplary embodiment of the invention.

FIG. 2 is a diagrammatical illustration of a particle detection system in accordance with an exemplary embodiment of the invention.

FIG. 3 is a diagrammatical illustration of a particle detection system employing an air shroud around the particle stream in accordance with an exemplary embodiment of the invention.

FIG. 4 is a flow chart illustrating a process for detecting a particle type within a particle stream in accordance with an exemplary embodiment of the invention.

FIG. 5 is a diagrammatic illustration of an experimental setup for temperature-dependent fluorescence in accordance with an exemplary embodiment of the invention.

FIG. 6 is a graphical representation of variation in fluorescence spectra of tryptophan biological particles with respect to temperature recorded by employing the experimental set up of FIG. 5.

FIG. 7 is a graphical representation of variation in fluorescence spectra of NADH biological particles with respect to temperature recorded by employing the experimental set up of FIG. 5.

FIG. 8 is a graphical representation of fluorescence spectra of different particle types with respect to different temperatures in accordance with an exemplary embodiment of the invention.

### SUMMARY

Embodiments of the invention are directed to methods and systems for detecting particles disposed in a detection area.

One exemplary embodiment of the invention is a system for detecting a particle disposed in a detection area. The system includes a light-emitting source for generating light. The light is directed at the particle. The system further includes a modulator configured to in-situ modulate at least one environmental parameter of the particle to alter a detectable response of the particle. The modulator provides an enhancement in detection selectivity of the particle in the presence of interfering particles and species. Further, the system includes a detector configured to detect alteration in the detectable response of the particle.

Another exemplary embodiment of the invention is a system for detecting an air-borne biological particle. The system includes a light source configured to emit radiation of determined wavelength, a detection area into which the air-borne biological particle is disposed. The detection area allows interaction of the air-borne biological particle with the light source. The air-borne biological particle yields a detectable response on interaction with the light source. The system further includes a modulator for varying at least one environmental parameter in the detection area to alter a detectable response from the air-borne biological particle, and a detector for detecting the alteration in the detectable response by the air-borne biological particle. The modulator provides an enhancement in detection selectivity of the particle in the presence of interfering particles and species.

Another exemplary embodiment is a method for detecting a particle. The method includes directing radiation to a particle stream disposed in a detection area, wherein the particle stream is configured to emit one or more detectable responses upon interaction with the radiation. The method further includes modulating one or more environmental parameters inside the detection area to alter the one or more detectable responses, and detecting alteration in the one or more detectable responses. The modulating is carried out in-situ while detecting the alteration in the one or more detectable response. The modulating provides an enhancement in detection selectivity of the particle in the presence of interfering particles and species.

These and other advantages and features will be more readily understood from the following detailed description of preferred embodiments of the invention that is provided in connection with the accompanying drawings.

### DETAILED DESCRIPTION OF EXEMPLARY EMBODIMENTS

Generally, optical systems are employed to detect particles present within an environment enclosed in a detection area. Fluorescence spectroscopy is applied for detection of biological particles via analysis of native fluorescence of biomaterials known also as biofluorescence or autofluorescence. The fluorescence-based techniques generate data from certain molecular components of biological particles. Unfortunately, the intrinsic fluorescence bands from biological particles are relatively spectrally wide; the primary fluorophores in the majority of bioaerosols fall into only a few broad categories. As used herein, the term “particle” refers to any individual mass or collection of masses that can interact with energy, such as electromagnetic energy, to produce signature optical signals. The particles may be of varying scale. For example, the particles may be at an atomic scale or a molecular scale. At a larger scale, the particles may be a combination of molecules forming a spore, a virus, or a cell. For example, the biological particles may include a biological fluorophore.

The categories of biological fluorophores include the aromatic amino acids, proteins, tryptophan, tyrosine, phenylalanine, nicotinamide adenine dinucleotide compounds, flavins, chlorophylls, or combinations of two or more thereof. In an exemplary embodiment, the biological fluorophores may include proteins. For example, the biological fluorophores may include tryptophan, riboflavin, a nicotinamide adenine dinucleotide compound, or a combination of two or more thereof. Biological particles containing these fluorophores include biological spores, vegetative bacteria, proteins, DNA, viruses, toxins, and fragments of these particles.

Embodiments of the invention relate to a method for enhancement of discrimination of biological particles by modulating one or more environmental parameters. In one embodiment, fluorescence and/or phosphorescence signatures of the particles may be compared with the reference signatures. In an exemplary embodiment, variation in the detectable response of the biological particles may be compared with a reference calibration curve to identify the biological particles. In certain embodiments, nicotinamide adenine dinucleotide hydrogen (NADH), indicative of biological activity or viability, may be coupled with information about fluorescence properties of other biological particles to detect the other biological particles. For example, differences in emission properties of the other biological particles may be identified as a function of different environmental conditions and these differences may be measured and applied for selective discrimination between NADH and other biological particles.

For such discrimination enhancement, the fluorescence and phosphorescence may be applied as a function of various environmental parameters as will be described in detail below. Such detection capability is useful in environmental monitoring, especially for hazardous biological particles for civilian and military requirements.

The particles, such as biological particles, which are to be detected may be air-borne, or dispersed in an aqueous medium inside the detection area. The particles may be detected by modulating one or more environmental parameters around the particles. Non-limiting examples of the environmental parameters may include a temperature, an electric field, a magnetic field, gravity, acceleration, a pressure, an exposure time, a moisture content, a chemical composition, or a combination of two or more thereof. For example, the temperature of the particles may be modulated by modulating the temperature of the gaseous environment in which the particles are disposed. In one embodiment, the temperature of the environmental parameters may be varied in a range of from about −4° C. to about 95° C. In another embodiment, the temperature of the environmental parameters may be varied from about 0° C. to about 90° C. In one embodiment, the temperature of the environmental parameters may be varied between room temperature to about 90° C. or higher. In yet another embodiment, the temperature of the environmental parameters may be varied in a range of from about −98° C. to about 95° C. Similarly, the pressure on the particles may be modulated by changing the pressure of the gaseous environment in which the particles are disposed. The exposure time refers to the time for which the particles are exposed to the light emitted by the light-emitting source. As will be described in detail below, the exposure time may be varied depending on the type of particles. In one embodiment, the chemical composition may be varied by varying the oxygen, or the moisture content of the environment around the particles.

In certain embodiments, a detectable response comprises signal intensity, emission spectra, excitation spectra, emission lifetime, absorption spectra, thermal emission, signal reversibility, electronic absorption spectra, electronic emission spectra, vibrational spectra, rotational spectra, Raman, surface-enhanced Raman, infrared, electromagnetic radiation, polarization property, bleaching rate, or a combination of two or more thereof. In an exemplary embodiment, certain chemicals may be able to restore original signal intensity after switching back to the starting temperature, but proteins may not be able to restore their original intensity after switching back to the original temperature because heating above 37° C. may denature the proteins. For example, as will be appreciated, emission spectra from fluorescence decays of NADH at different temperatures have distinctly different components. Although the decays are well described by four well-separated components, only two of those make a significant contribution to the kinetics. In an exemplary embodiment, the average fluorescence lifetime of NADH in solution is 0.39 nanoseconds at 20° C. The first and second decay components are 0.3 nanoseconds and 0.7 nanoseconds at 10° C., 0.28 nanoseconds and 0.62 nanoseconds at 20° C., and 0.24 nanoseconds and 0.55 nanoseconds at 40° C. with also changing pre-exponential factors. The pre-exponential factors reflect the frequency with which the system successfully passes through the transition state with the change in environmental parameters. Moreover, the temperature dependence of NADH fluorescence is the result of two simultaneous processes: (1) a shift of the lifetime amplitudes from the long to the short component when the temperature is increased, and (2) an Arrhenius dependency of both components with similar activation energies of about 1.5 kcal/mol. This two-process temperature dependence of NADH fluorescence provides a tool for biological particle discriminations. Indeed, other biological particles/species that will interfere with NADH measurements may be discriminated against NADH fluorescence by performing the measurements at different temperatures. For example, the indole groups of tryptophan residues are the dominant sources of UV absorbance and emission in proteins. In one embodiment, the differences between the temperature dependence of tryptophan alone and tryptophan within BSA may be employed in the detection. In another example, the differences between the temperature dependence of fluorescence of NADH and flavin may be employed in the detection.

In one embodiment, the temperature may be modulated by employing even low-cost techniques. For example, a thermoelectric heating/cooling may be employed for sensor applications for samples disposed in micro channels. The heating system provides a desired rapid change in temperature, thereby changing the response of the biological particles. In one embodiment, the cooling system may be enabled by applying the heating system in an opposite electrical polarity, thereby cooling the biological particles. In another embodiment, a supersonic expansion approach may be applied for cooling. In this embodiment, a stream of particles is forced through a small opening. Upon a release through the opening, the particles cool down. In an exemplary embodiment, at low temperatures the emission bands of the biological particles may narrow down. Such spectral features facilitate the determination of biological particles.

Additionally, in certain embodiments, the effect of the gas composition and moisture levels on the fluorescence and phosphorescence intensity of the particles may be used.

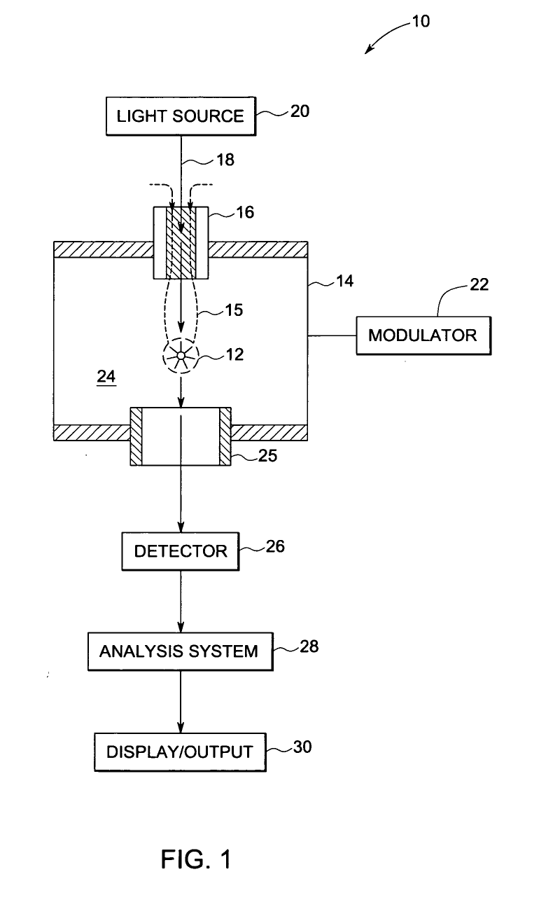

Referring now to FIG. 1, a particle detection system 10 for detecting particles 12 is illustrated. For ease of description, the particles 12 will be described herein as being biological in nature. The biological particles 12 may be disposed in an enclosed container 14. The biological particles 12 may be introduced inside the container 14 in the form of a particle stream 15. In one embodiment, a filter may be employed to filter the air stream before entering the container 14. The container 14 may include a passageway 16 to guide a light 18, from a light source 20, and/or a particle stream 15 to reach the biological particles 12. It should be appreciated that any suitable light-emitting source 20 may be utilized, such as, for example, light emitting diodes, including surface-emitting light emitting diodes, ultraviolet light emitting diodes, edge-emitting light emitting diodes, resonant cavity light emitting diodes, flip-chipped light emitting diodes, gas-discharge lamps, mercury lamps, filament lamps, black-body radiators, chemo-luminescent media, organic light emitting diodes, phosphor upconverted sources, plasma sources, solar radiation, sparking devices, vertical light emitting diodes, and wavelength-specific light emitting diodes, lasers, and laser diodes, and any other suitable light-emitting device capable of emitting a sufficiently high intensity light of the desired wavelength. “Sufficiently high intensity light” means sufficient intensity to induce an effective optical signal, such as particle fluorescence. The term “wavelength” should be understood to encompass a range of wavelengths and to refer to a spectral range of electromagnetic energy. It should be noted that the direction of light 18 from the light source 20 may be from any angle, including orthogonal to the direction of particle flow. Furthermore, the light-emitting source 20 may be pulsed to achieve the desired intensity of light without sacrificing reliability or lifetime. Another advantage of a very fast pulsed source, such as an LED, would be to synchronize the detector to the light source 20 for the purpose of improving the signal to noise ratio. A heat sink may be attached to the light-emitting source 20 to enhance heat dissipation.

The container 14 may include reflective coatings on the interior or the exterior of the container 14. For example, a reflective coating may be disposed on an inner surface (meaning a surface facing the interior 24 of the enclosure 14), thus serving to reflect any light striking such surface from within the enclosure 14. Alternatively, a reflective coating may be disposed on an outer surface (meaning a surface facing away from the interior 24 of the enclosure 14), thus serving to refract any light striking from within the enclosure 14.

The container 14 may include materials such as glass, quartz, silica, TEFLON®, amorphous fluoropolymer (TEFLON AF®), polycarbonate, or a combination of two or more thereof. In one embodiment, the container may include a substrate material that may be coated a film. The biological particles 12 inside the container 14 may be disposed in a gaseous environment. For example, the biological particles inside the container 14 may be air-borne. The biological particles 12 may be introduced in the container along with a particle stream. Further, air stream or other gases may be introduced into the container 14. In one embodiment, a pump may be provided to render a pressure differential necessary to pull both the particle stream and the air stream into the interior 24 of the container.

The system 10 may further include a modulator 22 in operative association with the container 14. The modulator 22 is configured to in-situ modulate one or more environmental parameters of the biological particles 12 in the container 14 to alter a detectable response of the biological particles. For example, the modulator may be configured to change the temperature of the biological particles 12 enclosed in the container 14. As used herein, the term “in-situ” refers to the modulation of the environmental parameters of the container 14 without having to stop the working of the container 14. For example, the temperature of the environmental parameters in the container 14 may be increased and simultaneously the change in the fluorescence spectra of the biological particles 12 may be captured. The modulator provides an enhancement in detection selectivity of the biological particles in the presence of interfering particles and species. Subsequently, the particle stream 15 having the biological particles 12 may be let out of the system via the outlet 25.

Further, the system 10 includes a detector 26 to detect the signals emitted by the biological particles 12. The detector 26 may be a photoconductor, a photodiode, a photomultiplier tube, an avalanche photodiode, or any photo detector capable of detecting single photons or collections of single photons, or a combination of two or more thereof. For example, the detector 26 may include CCD imagers, or spectral imagers. The detector 26 is configured to detect an alteration in the detectable response from the biological particles 12.

Further, the system 10 includes an analysis system 28, which receives signals from the detector 26 and conveys the signals to an output device 30. The analysis system 28 may be an univariate analysis system, or a multivariate analysis system. Where the optical spectrum comprises several wavelengths or an entire spectrum over a certain range, the optical characteristics of the sensing film may be determined using multivariate calibration algorithms such as Partial Least Squares Regression (PLS), Principal Components Regression (PCR), and the like. Given a large enough span of calibration samples, multivariate calibration models are generally more robust than univariate models due to enhanced outlier detection capabilities and increased tolerance toward slight shifting in peak position or band shape. Also, multivariate calibration models allow for measurement of more than one variable or component of interest in the particle stream. PLS models correlate the sources of variation in the spectral data with sources of variation in the sample. Preferably, the PLS model is validated by statistical techniques. Such statistical techniques include, but are not limited to, leave one out cross-validation, Venetian blinds, and random subsets. As will be recognized by those of ordinary skill in the art, all or part of the steps in the analysis of response of optical signals from the particle stream may be coded or otherwise written in computer software, in a variety of computer languages including, but not limited to, C, C++, Pascal, Fortran, Visual Basic®, Microsoft Excel, MATLAB®, Mathematica®, Java, and the like. Accordingly, additional aspects of the invention include computer software for performing one or more of the method steps set forth herein. The software code may be compiled and stored in executable form on computer readable media as, for example, computer ROM, floppy disk, optical disk, hard disks, CD ROM, or the like.

In one embodiment, multivariate analysis may be applied between two or more biological particles with very similar fluorescence emission spectra. In this embodiment, the fluorescence spectra may be based on the temperature modulation. The temperature modulation may be achieved by computer simulation. Subsequently, fluorescence spectra of the two or more biological particles may be collected at at least two temperatures. In one embodiment, the temperature-dependent temperature coefficients may be used to extrapolate the spectral profiles of mixtures of the two or more biological particles at different temperatures. Results of the multivariate analysis of each of the two or more individual biological particles and the combination of the two or more biological particles may be then illustrated. Such results may be illustrated using known pattern recognition tools.

The output device 30 may include a display or printer, to output the signatures generated during operation of the system 10. Displays/printers 30, analysis system 28, and similar devices may be local or remote from the system 10. For example, these interface devices may be positioned in one or more places within a lab, institution, or in a different location. Therefore, the interface devices may be linked to the system 10.

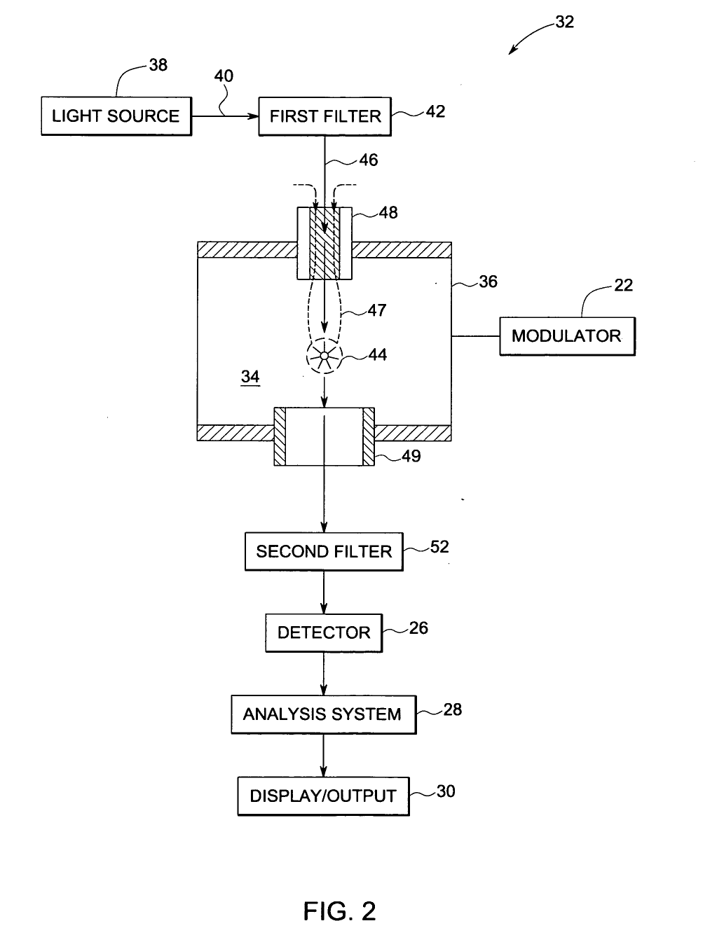

Referring now to FIG. 2, an alternate particle detection system 32 is illustrated. The illustrated system 32 includes a detection area 34 defined by the walls of container 36. The system 32 employs a light source 38 to generate light 40, which is then filtered by using a first filter 42. In one embodiment, the first filter 42 may be employed to filter out some of the wavelengths of the light 40 which may not be required to interact with the biological particles 44 to obtain the signature of the biological particles 44. A particle stream 47 having the biological particles 44 and the resultant light 46 may then be led into the container 36 via a passageway 48. A detector 26 may detect the light 46 after interaction with the biological particles 44. The particle stream 47 having the biological particles 44 may be let out of the system via an outlet 49. In the illustrated embodiment, a second optical filter 52 may be positioned between the outlet 49 and the detector 26. The optical filter 52 may filter out specific wavelengths, thus serving to eliminate one or more portions of the light spectrum to decrease the noise to signal ratio and also to prevent signals from biological particles which are not needed to be detected. Further, the system 32 may include the analysis system 28 and the output 30.

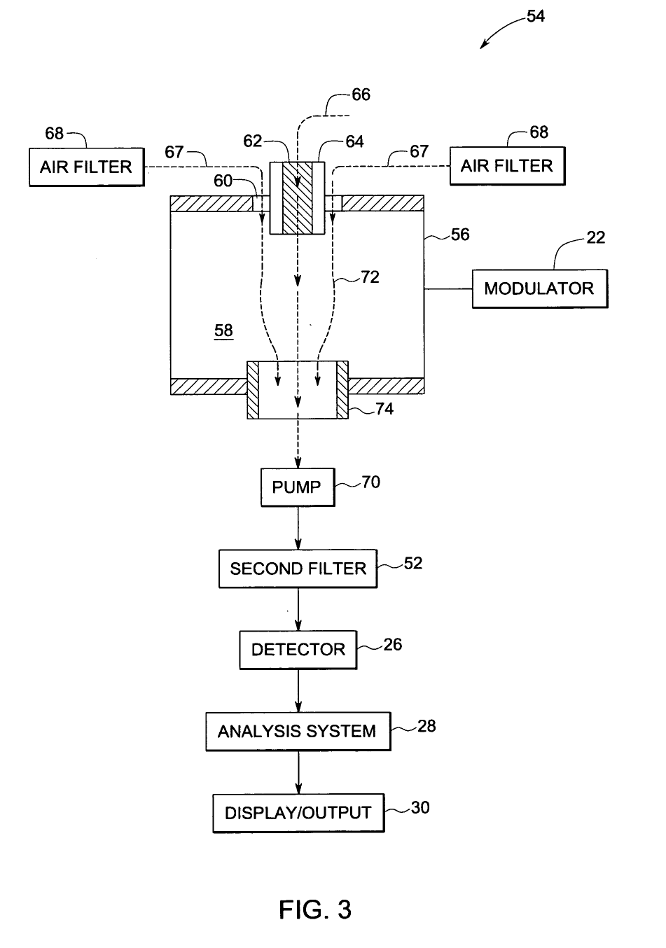

Turning now to FIG. 3, an alternate particle system 54 is illustrated. The system 54 includes an enclosure or container 56 having an interior space or detection area 58. The container 56 further includes an air inlet 60, which is concentric with an opening 62 of a particle inlet 64, which is attached to the enclosure 56. The opening for the air inlet 60 may be smooth-walled or they may be grooved to provide a spiral flow of air through the air inlet 60 and into the interior 58 of the enclosure 56. In other embodiments, the air inlet 60 may be nonexistent and another optically transparent conduit (not shown) may be utilized to segregate the particle stream 66 from the remaining environment of the interior 58 of the enclosure 56. Particles are introduced into the interior 58 of the enclosure within a particle stream 66. Air is introduced into the interior 58 of the enclosure 56 by passing an air stream 67 through an air filter 68 to filter the air stream 67. Filtering the air stream 67 reduces the likelihood of particulates from the air stream causing an erroneous fluorescence signature for the particle stream 66.

The container 56 further includes a pump 70 to provide the pressure differential necessary to pull both the particle stream 66 and the air stream 67 into the interior 58 of the enclosure 56. Various factors are taken into account to enable the air stream 67 extending into the interior 58 of the enclosure 56 to serve as an air-sheath 72 to the particle stream 66. Specifically, the pumping power of the pump 70, the distance into the interior 58 that the particle inlet 64 extends, the initial velocity of the particle stream 66, the size of the particle inlet 64, and the size of the sheath flow inlet or air inlet 60 all may be manipulated to ensure that the total flow of the air-sheath 72 is sufficiently less than the total flow of the particle stream 66 within the interior 58 to fully enshroud the particles within the particle stream 66. Nonetheless, the velocity of the air-sheath 72 is greater than the velocity of the particle stream 66. The difference in the velocities of the air-sheath 72 and the particle stream 66 within the interior 58 creates a pressure differential causing the particle stream 66 to remain within the air-sheath 72. Further, one or more of the various factors are manipulated to ensure that the particle stream 66 has no turbulent flow within the air-sheath 72. If either the velocity of the flow of air constituting the air-sheath 72 or the velocity of the radially inner particle stream 66 is too high, turbulence may be induced. Turbulence may coat the optical components of the particle detection system 54 and destroy optical sensitivity.

The air-sheath 72 serves as an optically transparent conduit serving to isolate the particle stream 66 from the remainder of the interior 58. It should be appreciated that other optically transparent conduits may be utilized to isolate the particle stream 66, such as, for example, poly ether ether ketone (PEEK), Teflon AF, fused silica, quartz, sapphire, or other transparent, low auto-fluorescent media capable of being formed into a conduit. The sheath also helps guide the particles and keeps the particles in the optical excitation path. Further, the sheath also helps inhibit the contamination of the particles disposed in the detector.

As the air-sheath 72 and the particle stream 66 extend closer to the pump 70, the air-sheath 72 begins to collapse radially inwardly toward the particle stream 66, and both streams 66, 72 exit the interior 58 through an outlet 74, which is in fluid connection with the pump 70. Through the use of the air-sheath 72, the particle stream 66 is isolated from the environment through an optically transparent mechanism, thereby enabling a more accurate optical measurement of particles within the particle stream 66. An additional benefit of the air-sheath 72 is that it can assist in cleaning the interior walls of the enclosure 56. Further, by ramping up the pump 70 intermittingly, a turbulent regime can be initiated to clean the interior 58 of the system 54. Optionally, ultrasonic waves may be used to clean the interior walls of the enclosure 56.

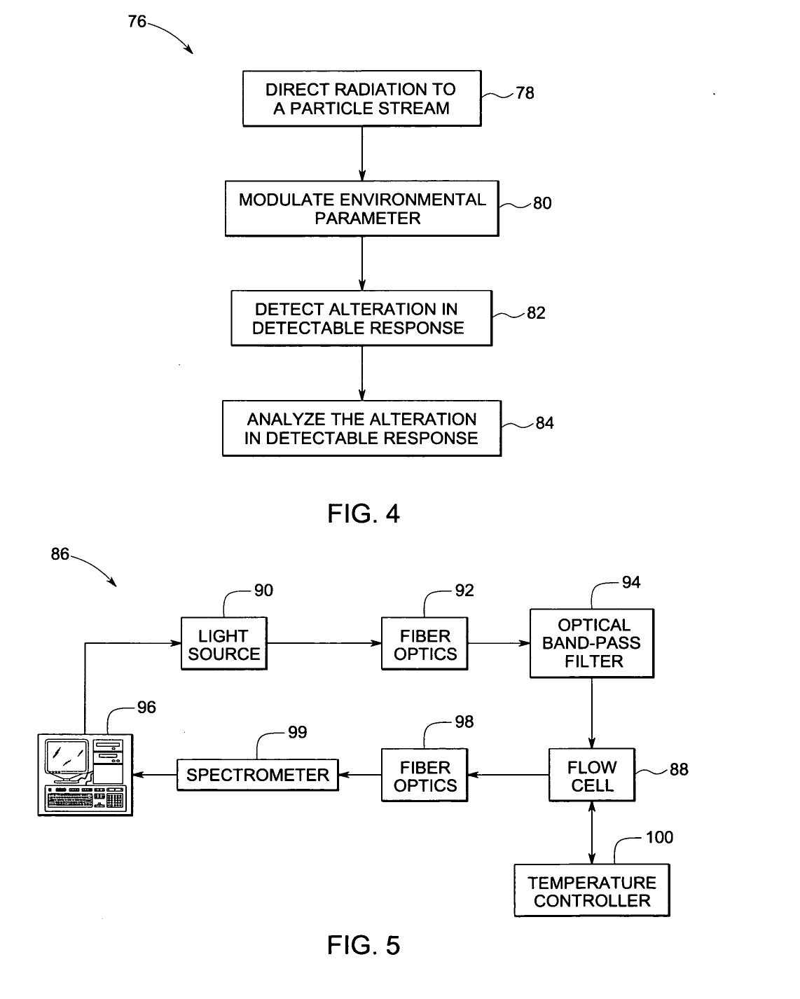

FIG. 4 illustrates a flow chart 76 for a method for detecting a particle, such as a biological particle. At step 78, radiation is directed to a particle stream disposed in a detection area. The radiation may include ultraviolet (UV) radiation, infrared (IR) radiation, visible radiation, or a combination thereof. The particle stream is configured to emit one or more detectable responses upon interaction with the radiation. For example, the particle stream may be configured to fluoresce or phosphoresce upon interaction with the radiation, such as UV radiation. At step 80, one or more environmental parameter of the particle stream may be modulated to alter one or more detectable responses from the particle stream. For example, the temperature of the particle stream may be increased. At step 82, the alteration in the response from the particle stream may be detected. For example, the emission spectra from the particle stream may be recorded at different temperature values. Subsequently, at step 84, the responses may be analyzed by using an analysis system, such as analysis system 28 (FIG. 1).

### EXAMPLE

An experimental setup 86 is illustrated in FIG. 5. The setup 86 includes a linear flow cell 88 with modifiable temperature. The flow cell 88 includes a sample placed in a cuvette. The sample includes tryptophan obtained from Sigma-Aldrich (Sigma-Aldrich Corporate Offices, 3050 Spruce St. Louis, Mo. 63103), NADH obtained from Sigma-Aldrich (Sigma-Aldrich Corporate Offices, 3050 Spruce St. Louis, Mo. 63103), commercially available diesel fuel, commercially available olive oil, commercially available canola oil, commercially available vegetable oil, e-coli obtained from American Type Culture Collection (ATCC P.O. Box 1549, Manassas, Va. 20108), riboflavin obtained from Sigma-Aldrich (Sigma-Aldrich Corporate Offices, 3050 Spruce St. Louis, Mo. 63103), and bovine serum albumin (BSA) obtained from Sigma-Aldrich (Sigma-Aldrich Corporate Offices, 3050 Spruce St. Louis, Mo. 63103). A Hamamatsu Lighting-Cure LC5 UV lamp 90 obtained from Hamamatsu (Bridgewater, N.J. 08807, U.S.) is used as the excitation light source. Excitation light is transmitted through fiber optics 92 obtained from Ocean Optics Inc. (830 Douglas Ave. Dunedin, Fla. 34698). Subsequently, the excitation light is filtered through optical band-pass filters 94 obtained from Melles Griot (55 Science parkway, Rochester, N.Y. 14620) and Omega Optical, Inc. (Delta Campus, Omega Drive, Brattleboro, Vt. 05301). The excitation light is filtered prior to the exposure to the sample in the flow cell 88. The flow cell 88 employed a TLC 50F Fiber-optic cuvette holder obtained from Quantum Northwest, Inc. (9723 W. Sunset Highway, Spokane, Wash. 99224-9426). The sample is exposed to the filtered excitation light at various temperature settings. Fluorescence light from the sample is collected through a fiber optic 98 disposed in orthogonal position relative to the excitation light and coupled to an Ocean Optics spectrometer 99 obtained from Ocean Optics Inc. (830 Douglas Ave. Dunedin, Fla. 34698) to gather spectral and intensity information. In case of strong scattering, additional optical filter may be used to prevent scattered excitation light to get into the spectrometer 99. Further, the setup 86 may also employ a computer 96 to control a lamp shutter (not shown). The lamp shutter regulates the exposure of the sample to the excitation light and the collection of the optical spectra from the sample at regular intervals. The regulation of exposure of the sample to the excitation light prevents possibilities of bleaching of the sample by over-exposure to the excitation light. The control shutter opens at a frequency of 1 opening per minute. The control shutter may be adjusted to different frequency intervals depending on the desired exposure of the sample and the intensity of the fluorescence light from the sample. The computer 96 employs a LabVIEW software to control the lamp shutter and collects fluorescence spectra every minute. Further, the computer 96, is coupled to spectrometer 99 via a USB connection. The flow cell 88 is also coupled to a temperature controller 100 obtained from Quantum Northwest, Inc. (9723 W Sunset Highway, Spokane, Wash. 99224-9426). Accompanied software from the same company allows writing software script to select temperature range, ramp rate, time periods, etc.

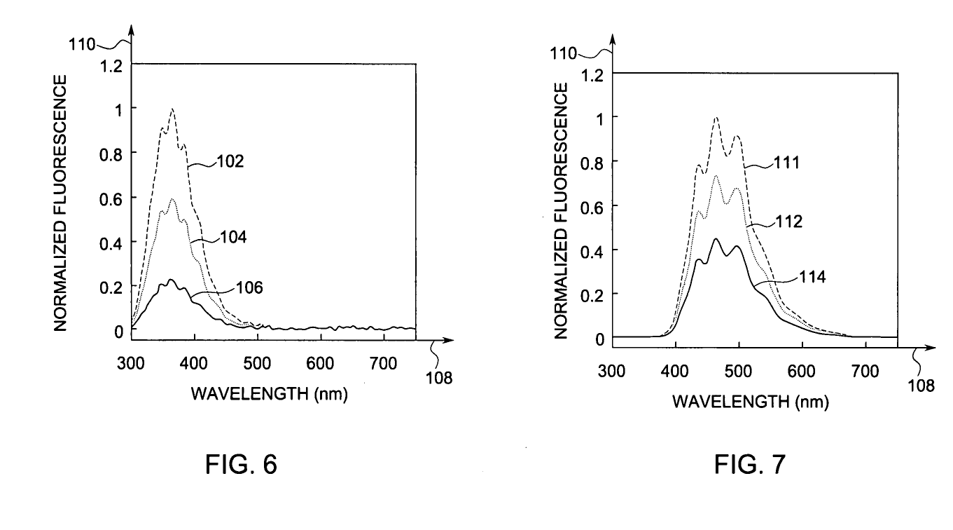

A first reading was taken while operating the flow cell 88 at 80° C. No fluorescence wavelength shift was observed at the first reading. Subsequently, the temperatures were varied between 20° C. and 80° C. The fluorescence intensity of most biological fluorophores: tryptophan, NADH, and riboflavin, showed a negative temperature dependency as illustrated in FIGS. 6 and 7. However, the non-biological particle of diesel fuel showed non-significant temperature-dependence in tested temperature range.

FIG. 6 illustrates fluorescence spectra 102, 104 and 106 for tryptophan at temperatures of 35° C., 50° C. and 80° C., respectively. The x-axis 108 illustrates the wavelength and the y-axis 110 illustrates the normalized values of the fluorescence. As illustrated, the fluorescence intensity goes down with increase in temperature for tryptophan. FIG. 7 illustrates a similar graph for NADH. The fluorescence spectra 111, 112 and 114 are observed at temperatures of 35° C., 50° C. and 80° C., respectively. The fluorescence intensity of NADH goes down with temperature increase.

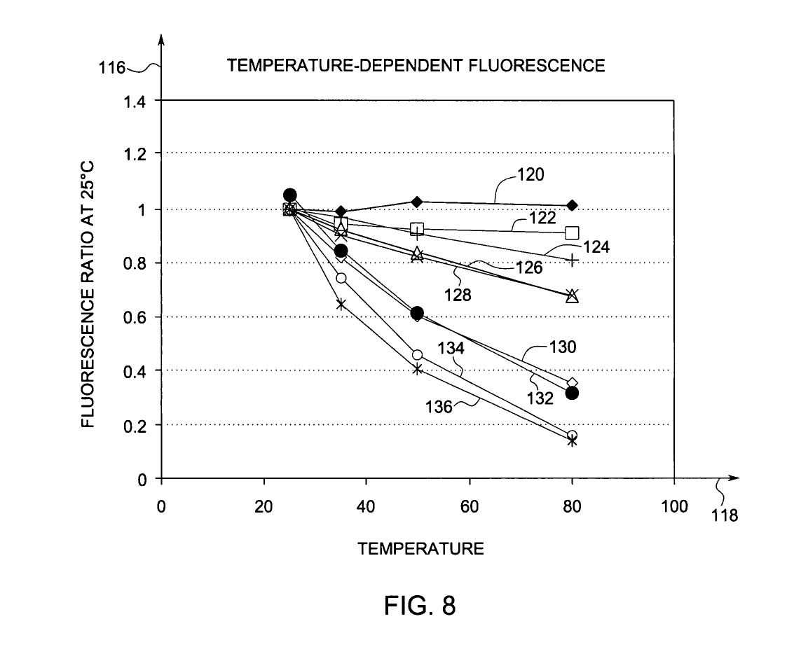

FIG. 8 illustrates fluorescence ratio (y-axis 116) of test samples at various temperatures (x-axis 118). The fluorescence ratio refers to the ratio of the fluorescence at that particular temperature and the fluorescence at temperature of 25° C. The temperature-dependence of the test samples illustrates varying degrees of thermal dependency of the samples with regard to temperature. This improves differentiation among species that fluoresce at similar spectral ranges. In the illustrated embodiment, the graphs represent the fluorescence ratio of the various samples at temperatures of 25° C., 35° C., 50° C. and 80° C. In the illustrated embodiment, change in fluorescence ratio is illustrated for diesel fuel 120, olive oil 122, riboflavin 124, vegetable oil mix 126, canola oil 128, NADH 130, BSA 132, tryptophan 134 and e-coli 136. As illustrated, the diesel oil (non biological particle) does not show change with temperature. For example, the main fluorophore of 132 BSA is tryptophan 134, therefore the fluorescence spectra of BSA 132 and tryptophan 134 are very similar. Therefore, heating to 35° C. is not adequate to differentiate BSA 132 and tryptophan 134, but at 50° C. detectable difference is observed in BSA 132 and tryptophan 134. Similarly, the temperature-dependence may be employed for samples whose fluorescence spectra are not similar but the detector and filter choices limit the differentiation between the two samples. For example, if a broad filter covering 430 nanometers to 630 nanometers range is selected for fluorescence measurements of NADH 130 and riboflavin 132 from vegetative bacteria. The 450 nanometers emission of NADH 130 is not distinguishable from 525 nanometers emission peak of riboflavin 132. However, fluorescence spectra of both NADH 130 and riboflavin 132 results at temperatures of 35° C. and 50° C. demonstrated differentiation at both temperatures.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include some of the described embodiments. Accordingly, the invention is not to be seen as limited by the foregoing description, but is only limited by the scope of the appended claims.

What is claimed as new and desired to be protected by Letters Patent of the United States is:

1. A system for detecting a particle disposed in a detection area,: comprising:
a light emitting source for generating light, wherein said light is directed at said particle;
a modulator configured to in-situ modulate at least one environmental parameter of said particle to alter a detectable response of said particle, wherein said modulator provides an enhancement in detection selectivity of said particle in the presence of interfering particles and species; and
a detector configured to detect alteration in said detectable response of said particle.

2. The system of claim 1, wherein said particle is air-borne.

3. The system of claim 1, wherein said particle is dispersed in an aqueous medium.

4. The system of claim 1, wherein said particle comprises a biological particle.

5. The system of claim 4, wherein said biological particle comprises a protein.

6. The system of claim 4, wherein said biological particle comprises tryptophan, tyrosine, riboflavin, a nicotinamide adenine dinucleotide compound, or a combination of two or more thereof.

7. The system of claim 1, wherein said at least one environmental parameter comprises a temperature, an electric field, a magnetic field, gravity, acceleration, a pressure, an exposure time, a moisture content, a chemical composition, or a combination of two or more thereof.

8. The system of claim 1, wherein said chemical composition comprises oxygen content.

9. The system of claim 1, wherein said detectable response comprises emission spectra, excitation spectra, emission lifetime, absorption spectra, thermal emission, signal reversibility, electronic absorption spectra, electronic emission spectra, vibrational spectra, rotational spectra, Raman, surface-enhanced Raman, infrared, electromagnetic radiation, signal intensity, polarization property, bleaching rate, or a combination of two or more thereof.

10. The system of claim 1, further comprises an analysis system in operative association with said detector.

11. The system of claim 10, wherein said analysis system comprises an univariate analysis system, or a multivariate analysis system.

12. The system of claim 1, wherein said light emitting source comprises light emitting diodes, surface-emitting light emitting diodes, ultraviolet light emitting diodes, edge-emitting light emitting diodes, resonant cavity light emitting diodes, flip-chipped light emitting diodes, gas-discharge lamps, mercury lamps, filament lamps, black-body radiators, chemo-luminescent media, organic light emitting diodes, phosphor upconverted sources, plasma sources, solar radiation, sparking devices, vertical light emitting diodes, wavelength-specific light emitting diodes, lasers, laser diodes, or a combination of two or more thereof.

13. The system of claim 1, wherein said detector comprises a photoconductor, a photodiode, a photomultiplier tube, an avalanche photodiode, or any photo detector capable of detecting single photons or collections of single photons, or a combination or array of two or more thereof.

14. The system of claim 1, further comprising a conduit through which a particle stream having said particle is transmitted into said detection area and a concentric air inlet through which an air-sheath is introduced into said detection area.

15. The system of claim 14, further comprising a pump configured to enable transmission of said particle stream through said detection area.

16. The system of claim 1, wherein said particle fluoresces or phosphoresces on interaction with said light.

17. A system for detecting an air-borne biological particle, comprising:
a light source configured to emit radiation of determined wavelength;
a detection area into which said air-borne biological particle is disposed, wherein said detection area allows interaction of said air-borne biological particle with said light source, and wherein said air-borne biological particle yields a detectable response on interaction with said light source;
a modulator for varying at least one environmental parameter in said detection area to alter a detectable response from said air-borne biological particle, wherein said modulator provides an enhancement in detection selectivity of said particle in the presence of interfering particles and species; and
a detector for detecting said alteration in said detectable response by said air-borne biological particle.

18. The system of claim 17, wherein said air-borne biological particles comprise a protein.

19. The system of claim 17, wherein said air-borne biological particles comprise tryptophan, tyrosine, riboflavin, a nicotinamide adenine dinucleotide compound, or a combination of two or more thereof.

20. The system of claim 17, wherein said at least one environmental parameter comprises a temperature, an electric field, a magnetic field, gravity, acceleration, a pressure, an exposure time, a moisture content, a chemical composition, or a combination of two or more thereof.

21. The system of claim 17, wherein said detectable response comprises emission spectra, excitation spectra, emission lifetime, absorption spectra, thermal emission, signal reversibility, electronic absorption spectra, rotational spectra, electronic emission spectra, vibrational spectra, Raman, surface-enhanced Raman, infrared, electromagnetic radiation, signal intensity, polarization property, bleaching rate, or a combination of two or more thereof.

22. A method for detecting a particle, comprising:
directing radiation to a particle stream disposed in a detection area, wherein said particle stream is configured to emit one or more detectable responses upon interaction with the radiation;
modulating one or more environmental parameters inside the detection area to alter the one or more detectable responses, wherein said modulating provides an enhancement in detection selectivity of the particle in the presence of interfering particles and species; and
detecting alteration in the one or more detectable responses;
wherein said modulating is carried out in-situ while detecting the alteration in the one or more detectable response.

23. The method of claim 22, wherein the particle stream comprises air.

24. The method of claim 22, wherein the fluid comprises an aqueous medium.

25. The method of claim 22, wherein said environmental parameters comprise a temperature, a pressure, a moisture content, a gas composition, electric field, magnetic field, gravity, acceleration, or a combination of two or more thereof.

26. The method of claim 25, wherein said modulating comprises changing the temperature of the particle stream from about −4° C. to about 95° C.

27. The method of claim 22, further comprising filtering the radiation prior to the interaction of the radiation with the particle stream.

28. The method of claim 22, wherein said detecting comprises detecting fluorescence or phosphorescence of the particle stream.

29. The method of claim 22, further comprising analyzing the alteration in the one or more detectable response.

30. The method of claim 29, wherein said analyzing comprises univariate analyzing or multivariate analyzing.

31. The method of claim 20, further comprising comparing variation in the detectable response with a reference calibration curve.
